(19) United States
(12) Patent Application Publication
(10) Pub. No.: US 2007/0238807 A1
(43) Pub. Date: Oct. 11, 2007

(54) Water resistant film forming compositions incorporating hydrophilic activities

(76) Inventors: Adam L. Safir, Berkeley, CA (US); Damian Hajduk, San Jose, CA (US); Didier G. Benoit, San Jose, CA (US); Marcelo E. Piotti, Fremont, CA (US); Shaofeng Ran, Clarksburg, MD (US); Steven X. Zong, Santa Clara, CA (US)

Correspondence Address: Brooks & Cameron, PLLC, Suite 500, 1221 Nicollet Avenue, Minneapolis, MN, 55403, US

(21) Appl. No.: 11/732,238

(22) Filed: Apr. 3, 2007

Related U.S. Application Data
(60) Provisional application No. 60/789,757, filed on Apr. 6, 2006.

(51) Int. Cl. A61L 24/00

(52) U.S. Cl. 523/113

(57) ABSTRACT

Water Resistant Film Forming Compositions Incorporating Hydrophilic Activities A film forming composition that includes a polymer having from about 80 mole percent to about 100 mole percent of a hydrophobic component, and from about 0 to about 20 mole percent of a charged component; a bioactive agent; and a solvent in which the polymer and the bioactive agent are homogeneously dispersed in the composition. The film forming composition can be used as a “liquid bandage” to form a water resistant film on a biological surface, where the polymer and the bioactive agent remain miscible.

### CROSS-REFERENCE TO RELATED APPLICATIONS

This application claims the benefit of U.S. Provisional Application No. 60/789,757, filed Apr. 6, 2006, the entire content of which is incorporated herein by reference.

### TECHNICAL FIELD

The disclosure relates generally to water resistant film forming compositions for covering a biological surface and methods of forming the compositions.

### BACKGROUND

“Liquid bandages” continue to be a developing area of wound care. Liquid bandages typically include a polymer or a curable resin solution that, when applied to an injured site, dry to form a waterproof flexible film. The film acts to protect the injured site from the outside while retaining moisture at the injured site. Liquid bandages can also offer a cosmetic benefit that is especially attractive to adult consumers.

The polymer films formed by a liquid bandage may also serve to deliver an active agent (e.g., a therapeutic agent) to the injury site. However, incorporation of the active agent to the polymer solution has not always been successful. For example, incorporating an active agent into the polymer solution of the liquid bandage often leads to limitations on the films that include structural failure due to cracking and delamination from the injury site. Furthermore, they tend to be limited in terms of the range of active agents that can be used, the range of amounts of active agents that can be included within a delivery system, and the range of the rates at which the included active agents are delivered therefrom.

Another reason why incorporation of the active agent into the polymer solution has not always been highly successful is that many of the existing polymer based liquid bandage systems use hydrophobic polymers in order to provide an acceptable degree of “wash resistance.” The use of hydrophobic polymers, however, often times precludes the incorporation of active agents such as hydrophilic anaesthetics or hydrophilic antimicrobials, such as benzalkonium chloride, benzocaine or lidocaine, as these agents precipitate rapidly in such formulations.

Thus, there is a continuing need for active agent delivery systems with greater versatility and tunability, particularly when hydrophilic active agents are used with liquid bandage formulations.

### SUMMARY OF THE DISCLOSURE

Embodiments of the present disclosure provide film forming compositions having a polymer and optionally a bioactive agent in a solvent, where the compositions can be used for various applications, such as a “liquid bandage” to form a water resistant film for covering and protecting injuries to a biological surface.

In some embodiments of the present disclosure, the polymer includes both a hydrophobic component and a charged component that associate with the optional bioactive agent to allow the polymer and the bioactive agent to remain homogeneously dispersed in solution and upon evaporation of the solvent, to form a water resistant film. In one embodiment, the bioactive agent is hydrophilic and yet still remains homogeneously dispersed with the polymer in solution and upon film formation. As the bioactive agent remains miscible with the polymer in the liquid state and the film state, the bioactive agent can provide a therapeutic and/or cosmetic benefit to the biological surface. In addition, the film forming compositions of the present disclosure can retain these bioactive agents for delivery to a biological surface without compromising the mechanical characteristics, such as wash resistance, of the resulting film.

According to some embodiments of the disclosure, the film forming composition includes a polymer having from about 80 mole percent to about 100 mole percent of a hydrophobic component, and from about 0 to about 20 mole percent of a charged component. The film forming composition also optionally includes a bioactive agent and a solvent. In one embodiment, the polymer and the bioactive agent are substantially dispersed throughout the composition. For such embodiments, a useful solvent includes those typically having a boiling point less than 100° C. at 1 atm, more specifically having a boiling point less than 75° C. at 1 atm. In one embodiment, such a solvent includes about 90 to about 100 weight percent of one or more alkanes and about 0 to about 10 weight percent of one or more non-aqueous polar solvents.

In some embodiments, the polymer can have about 5 mole percent or less of methacrylic acid (MAA) for the charged component, where the remaining mole percent is 2-ethyl hexyl methacrylate (EHMA) and methyl methacrylate (MMA) for the hydrophobic component in a ratio of about 75/25 to about 50/50 mole percent EHMA/MMA. More specifically, the polymer can have about 2 mole percent or less of MAA for the charged component, while the remaining mole percent of EHMA and MMA for the hydrophobic component is in a ratio of about 55/45 to about 50/50 mole percent EHMA/MMA. A useful solvent for these embodiments can include about 90 to about 100 percent by volume of octane and about 0 to about 10 percent by volume of isopropanol. More specifically, the solvent is 95 to 99 percent by volume of octane to about 1 to 5 by volume of isopropanol. For such embodiments, octane is preferably the isomer isooctane.

In some embodiments, the polymer of the present disclosure has about 0 to about 5 mole percent of MAA for the charged component, and about 30 to about 45 mole percent of MMA, about 1 to about 15 mole percent of 2-methoxyethyl methacrylate (MEMA), with the remaining mole percent of the polymer being EHMA, where MMA, MEMA, and EHMA are the hydrophobic component. More specifically, the polymer comprises about 0 to 2 mole percent of MAA, about 35 to about 40 mole percent of MMA, about 5 to 15 mole percent of MEMA, with the remaining mole percent of the polymer being EHMA. A solvent for these embodiments can include about 85 to 100 percent by volume of octane and about 0 to 15 percent by volume of isopropanol. More specifically, the solvent is 90 to 99 percent by volume octane and 1 to 10 percent by volume of isopropanol, and even more specifically, the solvent is 95 to 99 percent by volume octane and 1 to 5 percent by volume of isopropanol. For such embodiments, octane is preferably the isomer isooctane.

In some embodiments, the polymer further includes a hydrophilic component, where the polymer comprises about 0 to about 10 mole percent of the charged component, with the remaining mole percent of the polymer being the hydrophilic component and the hydrophobic component in a ratio of about 60/40 percent to about 80/20 percent. In one embodiment, the polymer includes about 2.0 to about 7.5 mole percent of acrylic acid (AA) for the charged component, with the remaining mole percent being methyl acrylate (MA) for the hydrophobic component, and 2-hydroxyethyl methacrylate (HEMA) for the hydrophilic component in a ratio of about 40/60 percent MA/HEMA to about 20/80 percent MA/HEMA. In one embodiment, a solvent can include about 60 to 85 percent by volume of water and about 15 to 40 percent by volume of ethanol. More specifically the solvent includes about 65 to 75 percent by volume of water and about 25 to 35 percent by volume of ethanol, and even more specifically the solvent for this embodiment includes about 70 percent by volume of water and about 30 percent by volume of ethanol.

Additional embodiments of the present disclosure include polymers for making the film forming composition and methods of making the film forming compositions, as discussed herein.

As used herein, the terms “a,” “an,” “one or more,” and “at least one” are used interchangeably.

Unless otherwise indicated, all numbers expressing quantities of ingredients, reaction conditions, and so forth used in the disclosure and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

As used herein a “biological surface” includes, but is not limited to, skin, nails, and mucous membranes. As used herein “injuries” to a biological surface include, but are not limited to, cuts, tears, cracks, chapping, abrasions, blisters, incisions, lacerations, lesions, burns, or other wounds to the biological surface.

As used herein, “film forming composition” includes a liquid polymeric coating composition according to the present disclosure that may be cured and/or dried to leave a flexible film adhered to a surface, such as a biological surface. Some embodiments of the film forming compositions can be cured and/or dried at room temperature (20° C.-25° C.) when applied to the biological surface.

As used herein, “flexible” is meant to include that the films formed with the film forming compositions do not substantially crack in response to ordinary flexion of the biological surface.

As used herein a “bioactive agent” includes, but is not limited to, a therapeutic agent released to the underlying biological surface of the film and/or a cosmetic agent for aesthetic purposes. Examples of a “therapeutic agent” can include, but are not limited to, those therapeutically effective amounts of antimicrobials, topical anesthetics, antifungals, antioxidants, and mixtures thereof. Examples of a “cosmetic agent” can include, but are not limited to, pigments, dyes, and combinations thereof.

As used herein “polymer” or “polymeric composition” includes co-, ter- or multi-polymers having various types of configurations including random, block, graft, hyperbranched, or otherwise, and mixtures thereof. Polymerization of the monomers to form polymers of the present disclosure can be conducted according to conventional methods, such as by bulk polymerization or by semi-continuous polymerization.

“Optional” or “optionally” means that the subsequently described event or circumstance may or may not occur, and that the description includes instances where said event or circumstance occurs and instances where it does not. For example, the phrase “optionally substituted hydrocarbyl” means that a hydrocarbyl moiety may or may not be substituted and that the description includes both unsubstituted hydrocarbyl and hydrocarbyl where there is substitution.

The term “alkyl” as used herein refers to a branched or unbranched saturated linear (i.e., straight chain) hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups (i.e., cycloaliphatic) such as cyclopentyl, cyclohexyl and the like. Generally, although again not necessarily, alkyl groups herein contain 1 to about 12 carbon atoms. The term “lower alkyl” intends an alkyl group of one to six carbon atoms, preferably one to four carbon atoms. “Substituted alkyl” refers to alkyl substituted with one or more substituent groups, and the terms “heteroatom-containing alkyl” and “heteroalkyl” refer to alkyl in which at least one carbon atom is replaced with a heteroatom.

The term “alkane” as used herein refers to a branched or unbranched saturated hydrocarbon group typically although not necessarily containing 1 to about 24 carbon atoms, such as methane, ethane, n-propane, isopropane, n-butane, isobutane, t-butane, octane, decane, and the like, as well as cycloalkane groups such as cyclopentane, cyclohexane and the like. Generally, although again not necessarily, alkanes herein contain 1 to about 12 carbon atoms.

The term “alkenyl” as used herein refers to a branched or unbranched hydrocarbon group typically although not necessarily containing 2 to about 24 carbon atoms and at least one double bond, such as ethenyl, n-propenyl, isopropenyl, n-butenyl, isobutenyl, octenyl, decenyl, and the like. Generally, although again not necessarily, alkenyl groups herein contain 2 to about 12 carbon atoms. The term “lower alkenyl” intends an alkenyl group of two to six carbon atoms, preferably two to four carbon atoms. “Substituted alkenyl” refers to alkenyl substituted with one or more substituent groups, and the terms “heteroatom-containing alkenyl” and “heteroalkenyl” refer to alkenyl in which at least one carbon atom is replaced with a heteroatom.

The term “alkynyl” as used herein refers to a branched or unbranched hydrocarbon group typically although not necessarily containing 2 to about 24 carbon atoms and at least one triple bond, such as ethynyl, n-propynyl, isopropynyl, n-butynyl, isobutynyl, octynyl, decynyl, and the like. Generally, although again not necessarily, alkynyl groups herein contain 2 to about 12 carbon atoms. The term “lower alkynyl” intends an alkynyl group of two to six carbon atoms, preferably three or four carbon atoms. “Substituted alkynyl” refers to alkynyl substituted with one or more substituent groups, and the terms “heteroatom-containing alkynyl” and “heteroalkynyl” refer to alkynyl in which at least one carbon atom is replaced with a heteroatom.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing one to six, more preferably one to four, carbon atoms. The term “aryloxy” is used in a similar fashion, with aryl as defined below.

The term “allenyl” is used herein in the conventional sense to refer to a molecular segment having the structure —CH═C═CH₂. An “allenyl” group may be unsubstituted or substituted with one or more non-hydrogen substituents.

The term “aryl” as used herein, and unless otherwise specified, refers to an aromatic substituent containing a single aromatic ring or multiple aromatic rings that are fused together, linked covalently, or linked to a common group such as a methylene or ethylene moiety. The common linking group may also be a carbonyl as in benzophenone, an oxygen atom as in diphenylether, or a nitrogen atom as in diphenylamine. Preferred aryl groups contain one aromatic ring or two fused or linked aromatic rings, e.g., phenyl, naphthyl, biphenyl, diphenylether, diphenylamine, benzophenone, and the like. In particular embodiments, aryl substituents have 1 to about 200 carbon atoms, typically 1 to about 50 carbon atoms, and preferably 1 to about 20 carbon atoms. “Substituted aryl” refers to an aryl moiety substituted with one or more substituent groups, (e.g., tolyl, mesityl and perfluorophenyl) and the terms “heteroatom-containing aryl” and “heteroaryl” refer to aryl in which at least one carbon atom is replaced with a heteroatom.

The term “aralkyl” refers to an alkyl group with an aryl substituent, and the term “aralkylene” refers to an alkylene group with an aryl substituent; the term “alkaryl” refers to an aryl group that has an alkyl substituent, and the term “alkarylene” refers to an arylene group with an alkyl substituent.

The term “heteroatom-containing” as in a “heteroatom-containing hydrocarbyl group” refers to a molecule or molecular fragment in which one or more carbon atoms is replaced with an atom other than carbon, e.g., nitrogen, oxygen, sulfur, phosphorus or silicon. Similarly, the term “heteroalkyl” refers to an alkyl substituent that is heteroatom-containing, the term “heterocyclic” refers to a cyclic substituent that is heteroatom-containing, the term “heteroaryl” refers to an aryl substituent that is heteroatom-containing, and the like. When the term “heteroatom-containing” appears prior to a list of possible heteroatom-containing groups, it is intended that the term apply to every member of that group. That is, the phrase “heteroatom-containing alkyl, alkenyl and alkynyl” is to be interpreted as “heteroatom-containing alkyl, heteroatom-containing alkenyl and heteroatom-containing alkynyl.”

“Hydrocarbyl” refers to univalent hydrocarbyl radicals containing 1 to about 30 carbon atoms, preferably 1 to about 24 carbon atoms, most preferably 1 to about 12 carbon atoms, including branched or unbranched, saturated or unsaturated species, such as alkyl groups, alkenyl groups, aryl groups, and the like. The term “lower hydrocarbyl” intends a hydrocarbyl group of one to six carbon atoms, preferably one to four carbon atoms. “Substituted hydrocarbyl” refers to hydrocarbyl substituted with one or more substituent groups, and the terms “heteroatom-containing hydrocarbyl” and “heterohydrocarbyl” refer to hydrocarbyl in which at least one carbon atom is replaced with a heteroatom.

By “substituted” as in “substituted hydrocarbyl,” “substituted aryl,” “substituted alkyl,” “substituted alkenyl” and the like, as alluded to in some of the aforementioned definitions, is meant that in the hydrocarbyl, hydrocarbylene, alkyl, alkenyl or other moiety, at least one hydrogen atom bound to a carbon atom is replaced with one or more substituents that are functional groups such as hydroxyl, alkoxy, thio, phosphino, amino, halo, silyl, and the like. When the term “substituted” appears prior to a list of possible substituted groups, it is intended that the term apply to every member of that group. That is, the phrase “substituted alkyl, alkenyl and alkynyl” is to be interpreted as “substituted alkyl, substituted alkenyl and substituted alkynyl.” Similarly, “optionally substituted alkyl, alkenyl and alkynyl” is to be interpreted as “optionally substituted alkyl, optionally substituted alkenyl and optionally substituted alkynyl.”

As used herein all reference to the elements and groups of the Periodic Table of the Elements is to the version of the table published by the Handbook of Chemistry and Physics, CRC Press, 1995, which sets forth the new IUPAC system for numbering groups.

These and other features and advantages of the present disclosure are discussed in connection with illustrative embodiments of the disclosure below.

### DETAILED DESCRIPTION

Embodiments of the present disclosure provide a film forming composition that includes a polymer having a hydrophobic component and a charged component, and optionally a bioactive agent, where the polymer and the bioactive agent are substantially dispersed in a solvent to form the composition. Upon application to a surface, such as a biological surface, most or all of the solvent preferably evaporates leaving the polymer and the bioactive agent as an adherent conformable water resistant film. As the bioactive agent remains homogeneously dispersed with the polymer in both the liquid state and the film state, the bioactive agent can provide a therapeutic and/or cosmetic benefit to the biological surface.

In addition, film forming compositions of the present disclosure retain these bioactive agents for delivery to a biological surface without compromising the mechanical characteristics, such as the wash resistance, of the resulting film. For example, the films formed from the film forming composition embodiments are elastic, have good flexural and elongation durability, and are resistant to being washed off or swelling during exposure to soap and water. In addition, solvents used in the film forming composition are relatively quick-drying at room temperature, non-irritating to surfaces such as skin, and substantially painless (e.g., non-stinging) when applied to an injury on the biological tissue. The resulting film is also substantially non-water-sensitive, waterproof, non-tacky, does not attract and/or hold dirt, and can help to control water vapor and/or oxygen gas transmission there through.

As will be discussed herein, polymer embodiments can have unique properties resulting from the combination of the hydrophobic component and the charged component that allow for the bioactive agent to remain dispersed in the composition even after the evaporation of the solvent. According to various embodiments, the bioactive agent can remain miscible with the polymer even when the bioactive agent is hydrophilic. Not to be limited by theory, it is believed that the use of the charged monomers in the polymers of the present disclosure help solubilize the bioactive agents having a hydrophilic character that would not necessarily be soluble in the absence of the charged monomers.

Polymer embodiments of the present disclosure generally includes from about 80 mole percent to about 100 mole percent of a hydrophobic component, and from about 0 to about 20 mole percent of a charged component. The film forming composition also optionally includes a bioactive agent and a solvent in which the polymer and the bioactive agent are substantially dispersed in the composition. The hydrophobic component and the charged component of the polymer can be materials comprising a polymerizable monomer, oligomer, or polymer.

In additional embodiments, the polymer of the film forming composition can include a hydrophilic component, where the polymer has about 0 to about 10 mole percent of the charged component, and the remaining mole percent of the polymer is the hydrophilic component and the hydrophobic component in a ratio of about 60/40 percent to about 80/20 percent. The hydrophilic component of the polymer can be materials comprising a polymerizable monomer, oligomer, or polymer.

In some embodiments of the polymers of the disclosure, a combination of the hydrophobic and hydrophilic components may be used, either randomly or in separate blocks of the polymer (e.g., thermoplastic elastomers, grafts, etc). One variation in the selection of monomers to be used in the polymer can be using more than one monomer within each category of hydrophobic, charged or hydrophilic.

The term “hydrophobic component” is recognized in the art and is intended to include those monomers, generally alkyl acrylates, alkyl(meth)acrylates, (meth)acrylamide, allyl esters, or vinyl ester monomers, that include a hydrophobic moiety, e.g., esters having branched or unbranched, substituted or unsubstituted alkyl or cycloalkyl chains of generally one or more carbon atoms.

Examples of monomers for the hydrophobic component as provided herein include, but are not limited to, substituted and unsubstituted styrenes, isoprene, butadiene, vinyl acetate and esters of acrylic acid and methacrylic acid, where the ester group has a carbon atom chain of one or more carbon atoms, such as ethyl acrylate or methacrylate, propyl acrylate or methacrylate, butyl acrylate or methacrylate, and C₁₂ through C₁₈ esters of acrylic acid or methacrylic acid.

Hydrophobic components also can include alkyl esters, wherein the alkyl portion is a branched or unbranched, substituted or unsubstituted alkyl chain of 2 or more carbon atoms or a substituted or unsubstituted aryl group. Dialkyl and monoalkyl acrylamides and methacrylamides are further contemplated as hydrophobic components. Dialkyl methacrylamides and acrylamides include alkyl chains having at least 2 or more carbon atoms that can be substituted or unsubstituted, branched or unbranched. Monoalkyl methacrylamide or acrylamides include an alkyl chain having at least 4 carbon atoms or more that can be branched or unbranched, substituted or unsubstituted.

Suitable monomers for the hydrophobic component may be listed above and include, but are not limited to, vinyl chloride; vinylidene chloride; vinyl propionate; alpha-methylstyrene; t-butylstyrene; butadiene; cyclohexadiene; ethylene; propylene; vinyl toluene, t-butyl acrylate, t-butyl methacrylate, and mixtures thereof. Additional hydrophobic monomers include methyl acrylate (MA), methyl methacrylate (MMA), ethyl acrylate (EA), ethyl methacrylate (EMA), 2-ethyl hexyl methacrylate (EHMA), 2-methoxyethyl acrylate (MEA), 2-methoxyethyl methacrylate (MEMA), styrene (Sty), vinyl acetate (VA), vinyl neodecanoate (VND), 2-ethylhexyl acrylate (EHA), butyl acrylate (BA), n-butyl methacrylate (BMA), isobutyl methacrylate, lauryl acrylate (LA), and mixtures thereof.

As used herein the term “hydrophilic component” is intended to include those monomers, generally hydoxyalkyl acrylates, hydoxyalkyl(meth)acrylates, (meth)acrylamide, and vinyl ester monomers, that include a hydrophilic moiety, e.g., alkoxyesters having alkyl or cycloalkyl chains having one or more hydroxyl, polyethoxy, polypropyloxy groups attached thereto. Hydrophilic components will, in general, be insoluble in the nonpolar, low boiling point solvents useful for solvation of the hydrophobic polymer.

Examples of monomers for the hydrophilic component as provided herein include, but are not limited to N-vinyl pyrrolidone (VPL); 2-hydroxyethyl acrylate (HEA) or 2-hydroxyethyl methacrylate (HEMA); N-tris(hydroxymethyl) acrylamide or N-[tris(hydroxymethyl)methyl]acrylamide (THMMAM); N,N-dimethyl acrylamide (DMA); N-acryloyl or methacryloyl morpholine; “PEGylated” or “ethoxylated” acrylates or methacrylates, such as polyethyleneglycol acrylate (PEGA), polyethyleneglycol methacrylate (PEGMA), 2-(2-ethoxyethoxy)ethyl acrylate or methacrylate; N-isopropyl acrylamide or methacrylamide; glycerol mono acrylate or methacrylate; N-(2-hydroxypropyl)acrylamide or methacrylamide and N-(2-hydroxyethyl)acrylamide and methacrylamide; methacrylamide, N-t-butyl acrylamide, the half esters of maleic anhydride, acrylamide, acrylate alcohols, hydroxyethyl methacrylate, vinyl ethers (such as methyl vinyl ether), maleimides, other polar vinyl heterocyclics, allyl alcohol, vinyl alcohol (such as that produced by the hydrolysis of vinyl acetate after polymerization), and mixtures thereof.

In one embodiment, the hydrophilic monomers include N-vinylpyrrolidone (VPL), 2-hydroxyethyl acrylate (HEA), 2-hydroxylethyl methacrylate (HEMA), N,N-dimethyl acrylamide (DMA), N-[tris(hydroxymethyl)methyl]acrylamide (THMMAM), polyethyleneglycol acrylate (PEGA), polyethyleneglycol methacrylate (PEGMA), and mixtures thereof.

As used herein a “charged component” includes those monomers that contain a cationic or anionic charge associated with the molecule, or are not charged, but easily ionized (such as acrylic acid, methacrylic acid, and amine containing monomers). For example, the anions can include an ammonium ion, quaternary ammonium ions, halides, sulfate, carbonate, phosphate, phosphite, acetate and the like. Suitable cations include various metal ions such as sodium, potassium, calcium, etc.

In one embodiment, the monomers for the charged component may include, but are not limited to, N,N-dimethylaminoalkyl acrylate, such as N,N-dimethylaminoethyl acrylate (DMAEA), N,N-dimethylaminoalkyl methacrylate, such as quaternized dimethylaminoethyl methacrylate, [2(methacryloyloxy)ethyl]trialkylammonium salt, [2-(acryloyloxy)ethyl]trialkylammonium salt, such as [2-(acryloyloxy)ethyl]trimethylammonium chloride (AETMAC), N,N-dimethylaminoalkyl methacrylamide, N,N-dimethylaminoalkyl acrylamide, N,N,N-trimethylaminoalkyl methacrylamide salt, N,N,N-trimethylaminoalkyl acrylamide salt, (vinylbenzyl)trialklyammonium salt, such as (vinylbenzyl)trimethylammonium chloride (VBTMAC), diallyldimethylammonium chloride, acrylic acid (AA), methacrylic acid (MAA), itoconic acid (IC), crotonic acid, maleic anhydride, maleic acid, fumaric acid, mono-2-(methacryloyl)ethyl succinate (MAES), 2-acrylamido-2-methyl-1-propanesulfonic acid (AMPS), vinylphosphonic acid (VPA), vinyl imidazole, vinyl pyridine, styrene sulfonate, and mixtures thereof.

Polymers of the present disclosure may be prepared, for example, by free-radical polymerization methods, including but not limited to bulk, solution, emulsion, and suspension polymerization methods. For example, polymers of the present disclosure can be prepared by emulsion polymerization. According to the emulsion polymerization method, polymers suitable for use in the present disclosure are prepared by forming an emulsion comprising the desired monomers and a water-soluble radical initiator system (such as a thermal initiator or a photoinitiator) in an inert atmosphere such as nitrogen or argon, and then heating the emulsion until a reaction exotherm occurs. The reaction mixture is stirred and cooled and the resulting polymer is collected. Optionally, an ionic or nonionic surfactant may be added to the reaction mixture. Reducing agents may optionally be added to form an oxidation-reduction “Redox” pair with the radical initiator.

During the emulsion polymerization reaction, a quantity of the monomers can be charged batchwise or in a continuous or semicontinuous manner to the reaction vessel. By semi-continuous it is meant that a plurality of batches of the monomer is charged to the vessel during the course of the polymerization. The independent rate at which the monomers are added to the vessel will depend on the consumption rate of the particular monomer with time. Typically, the rate of addition of monomer will be about equal to the rate of consumption of monomer, i.e., conversion of monomer into polymer.

For the reaction, the reaction vessel can be initially charged with a solvent. Suitable solvents for polymerization reactions of the disclosure include, for example, water, ketones, ethers, polar aprotic solvents, esters, aromatic solvents, and aliphatic hydrocarbons, both linear and cyclic. Exemplary ketones include methyl ethyl ketone (2-butanone) (MEK), acetone, and the like. Exemplary ethers include alkoxyalkyl ethers, such as methoxy methyl ether or ethyl ether, tetrahydrofuran, 1,4 dioxane, and the like. Polar aprotic solvents include dimethyl formamide, dimethyl sulfoxide and the like. Suitable esters include alkyl acetates, such as ethyl acetate, methyl acetate, and the like. Aromatic solvents include alkylaryl solvents, such as toluene, xylene, and the like and halogenated aromatics such as chlorobenzene, and the like. Hydrocarbon type solvents include, for example, isooctane, hexane, cyclohexane, and the like.

In the case of emulsion polymerizations, a surfactant can be added, typically in an amount of 0.01% by weight to 1% by weight. The polymerization is usually initiated after an initial charge of monomer by adding an initiator or initiator system to the solvent phase.

The phrase “free-radical initiator,” within the context of the disclosure, refers broadly to compounds or mixtures of compounds that can lead to the formation of radical species under appropriate working conditions (e.g., thermal activation, irradiation, redox conditions, etc.). Examples of free-radical initiators useful in the polymerization mixture according to the present disclosure include alkyl peroxides, substituted alkyl peroxides, aryl peroxides, substituted aryl peroxides, acyl peroxides, alkyl hydroperoxides, substituted alkyl hydroperoxides, aryl hydroperoxides, substituted aryl hydroperoxides, heteroalkyl peroxides, substituted heteroalkyl peroxides, heteroalkyl hydroperoxides, substituted heteroalkyl hydroperoxides, heteroaryl peroxides, substituted heteroaryl peroxides, heteroaryl hydroperoxides, substituted heteroaryl hydroperoxides, alkyl peresters, substituted alkyl peresters, aryl peresters, substituted aryl peresters, percarbonates, halide compounds, and azo compounds, for example. Initiation may also be by heat or UV light, depending on the embodiment being practiced. Specific initiators can include lauroyl peroxide.

In various embodiments, the amount of initiator employed is typically between 0.03 and 2 weight %, or between 0.05 and 1 weight % of the total weight of the monomer mixture. The full amount of initiator may be added at the start of the polymerization or the initiator can be added to the polymerization in a continuous way during the polymerization process. A part of the initiator could also be added at the start and the remainder in one or more separate additional portions during the polymerization. Further addition(s) may be done batchwise or the further addition may be continuous. Emulsion polymerization system may further comprise auxiliaries, such as buffers and complex-formers.

Polymerization conditions that may be used include temperatures for polymerization typically in the range of from about 20° C. to about 110° C., more specifically from about 40° C. to about 90° C., and even more specifically from about 50° C. to about 80° C. The atmosphere may be controlled, with an inert atmosphere such as nitrogen or argon being useful, for example.

Polymerization conditions can also include the time for reaction, which may be, in various embodiments, from about 0.5 hours to about 72 hours, more specifically from about 1 hour to about 24 hours, and even more specifically from about 2 hours to about 12 hours. Conversion of monomer to polymer can also be at least about 50%, at least about 75% and even at least about 85%.

In another embodiment, polymers of the present disclosure may be obtained by a non-free-radical polymerization mechanism, such as the polymerization of monomers, such as oxazoline monomers shown below in Structure I, by a (living) cationic ring-opening polymerization process. In a copolymerization involving several oxazolines, random copolymers can be obtained by a cationic polymerization with different R substituants affecting the balance of hydrophobicity/hydrophilicity in the system. An example of a hydrophilic oxazoline is 2-methyl-2-oxazoline. A typical example of a hydrophobic oxazoline is 2-nonyl-2-oxazoline. Block copolymers can also be obtained by this living cationic ring-opening polymerization mechanism. A typical synthetic protocol of the synthesis of poly (2-nonyl-2-oxazoline) is reported by Hoogenboom et al, J. Comb. Chem. 2005, 7, 10-13, the disclosure of which is hereby incorporated by reference, involving the use of methyl tosylate as an initiator in a solvent mixture of acetonitrile and dichloromethane.

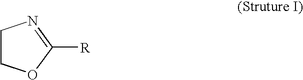

wherein R is selected from the group consisting of hydrogen, hydrocarbyl, substituted hydrocarbyl, heteroatom-containing hydrocarbyl, and substituted heteroatom-containing hydrocarbyl, and combinations thereof. More specifically, R may be selected from the group consisting of hydrogen, optionally substituted alkyl, optionally substituted aryl, optionally substituted alkenyl, optionally substituted alkynyl, optionally substituted allenyl, optionally substituted aralkyl, optionally substituted acyl, optionally substituted, aroyl, optionally substituted alkoxy, optionally substituted heteroalkyl, optionally substituted heteroaryl, and optionally substituted heterocyclyl.

The polymers of the present disclosure can have a structure selected from the group consisting of random copolymers, block copolymers, graft copolymers and hyperbranched copolymers. As used herein, “block copolymer” includes a polymer comprising at least two segments of differing composition; having any one of a number of different architectures, where the monomers are not incorporated into the polymer architecture in a solely statistical or uncontrolled manner. Although there may be three, four or more monomers in single block-type polymer architecture, it is considered herein as a block copolymer. Block copolymers can be prepared a number of ways, including sequential addition of monomers or using multi-functional control agents that may form a linking group between one or more blocks of the copolymers. Graft copolymers can be generated using initiators capable of initiating multiple free radical polymerizations under the controlled conditions of the present disclosure.

In yet another aspect of the present disclosure, the polymer can be a blend of polymers having hydrophobic components, charged components, and optionally hydrophilic components as provided herein. Alternatively, the polymer can be a blend of polymers, such as a polymer with hydrophobic components and charged components blended with a polymer having hydrophobic components, charged components and hydrophilic components.

The polymer may have molecular weights from 50,000 to several million in various embodiments. In one embodiment, the molecular weight range is 50,000 to 1,000,000 weight average molecular weight (M_{w}). In certain aspects of the disclosure, the Mw of the polymer is between about 65,000 and about 500,000, more specifically between about 75,000 and about 500,000, and more specifically between about 200,000 and 500,000. The M_{w} of the polymer may be controlled by varying initiator, initiator concentration, monomer concentration, reaction temperature, reaction solvent, and/or reaction method.

As provided herein, the types and mole fractions of monomers used to form polymers of the present disclosure can be selected to adjust permeability, adhesion, elasticity, flexibility, toughness, and temperature stability, among other film qualities, to meet the requirements of a liquid bandage. For example, at least one glass transition temperature (T_{g}) of the polymer can be adjusted to allow the polymer once in film form to be flexible from about 37° C. (e.g., body temperature) to about 50° C. The T_{g} of a monomer is a known parameter. When combinations of monomers are used in forming random copolymer the resulting T_{g} of the polymer is a function of the T_{g} of the different monomers. For example, polymers of the present disclosure can combine monomers with a high T_{g} and monomers with a low T_{g} to provide a polymer tuned for the desired T_{g} to prevent the resulting film from cracking when applied to the biological surface.

Screening and analysis of polymers suitable for the embodiments of the present disclosure can be accomplished through the use of parallel material processing techniques, among other techniques. For the present disclosure “Library Studio®” software (Symyx Technologies, Inc., Santa Clara, Calif., USA) can be used to design monomer libraries for the polymer of the present disclosure. In designing the libraries, varying proportions of different monomers for the hydrophobic component, charged component, and optionally the hydrophilic component, can be selected in an array for the different libraries. The resulting libraries can then optionally be stored in a “Renaissance Application Server®” (Symyx Technologies, Inc., Santa Clara, Calif., USA) from which information and instructions can be provided to “Impressionist®” software (Symyx Technologies, Inc., Santa Clara, Calif., USA) or by other instrument-control software to create and execute the library on an Automated Synthesis Station that synthesizes the polymers in an array format. The resulting polymers can then be screened for certain physical and functional properties, including chemical, solubility, thermal, and mechanical attributes.

In one embodiment, a polymer of the present disclosure is formed by copolymerizing about 5 mole percent or less of methacrylic acid (MAA) for the charged component, with the remaining mole percent of 2-ethyl hexyl methacrylate (EHMA) and methyl methacrylate (MMA) for the hydrophobic component in a ratio of about 75/25 to about 50/50 mole percent EHMA/MMA. In an additional embodiment, the polymer is formed by copolymerizing about 2 mole percent or less of MAA for the charged component, with the remaining mole percent of EHMA and MMA for the hydrophobic component in a ratio of about 55/45 to about 50/50 mole percent EHMA/MMA. This polymer demonstrates excellent adhesion to biological surfaces, is flexible and waterproof, is soluble in therapeutically compatible solvents such as isooctane and isopropanol, as will be discussed herein, and is very compatible with many kinds of bioactive agents, including those that are hydrophilic in nature.

In another embodiment, the polymer is formed by copolymerizing about 0 to about 5 mole percent of MAA for the charged component, about 30 to about 45 mole percent of MMA, about 1 to about 15 mole percent of 2-methoxyethyl methacrylate (MEMA), with the remaining mole percent of the polymer being 2-ethyl hexyl methacrylate (EHMA), where MMA, MEMA and EHMA are the hydrophobic component. In an additional embodiment, the polymer is formed by copolymerizing about 0 to 2 mole percent MAA, about 35 to about 40 mole percent MMA, about 5 to 15 mole percent MEMA, with the remaining mole percent of the polymer being EHMA. This polymer demonstrates excellent adhesion to biological surfaces, is flexible and waterproof, is soluble in therapeutically compatible solvents such as isooctane and isopropanol, as will be discussed herein, and is very compatible with many kinds of bioactive agents, including those that are hydrophilic in nature.

In another embodiment, the polymer is formed by copolymerizing about 2.0 to about 7.5 mole percent of acrylic acid (AA) for the charged component, the remaining mole percent of a methyl acrylate (MA) for the hydrophobic component, and 2-hydroxyethyl methacrylate (HEMA) for the hydrophilic component in a ratio of about 40/60 percent MA/HEMA to about 20/80 percent MA/HEMA. This polymer demonstrates excellent adhesion to biological surfaces, is flexible and waterproof, is soluble in therapeutically compatible solvents such as water and ethanol, as will be discussed herein, and is very compatible with many kinds of bioactive agents, including those that are hydrophilic in nature.

Embodiments of the present disclosure further include various methods of making the film forming composition that include dissolving the polymer of the present disclosure with a bioactive agent in a solvent. Solvents for use with the film forming compositions are desirably therapeutically safe and skin tolerant. Such solvents can have a sufficiently low boiling point (e.g., 75° C. or less) at 1 atmosphere that a coating of the film forming composition is touch dry within 3 minutes, within 2 minutes, and even within 1 minute, after being coated onto a biological surface at ambient temperature, pressure, and humidity (e.g., 22° C., 1 atmosphere and 50% RH). This can be achieved by monomer choice, molecular weight control, and solvent system choice. In one embodiment, the polymer of the present disclosure has a limited solubility in water (less than 1 percent by weight (wt %) in deionized water at 40° C.).

Suitable solvents for the film forming compositions include those in which the polymer is soluble at a concentration of at least 1 wt % at 25° C. Typically, the polymer content of film forming composition is no more than about 50 wt %, and/or no more than about 20 wt %, based on the total weight of the solution. In one embodiment, the polymer content of film forming composition is about 8 wt %, based on the total weight of the solution. In an additional embodiment, the film forming compositions can include a solvent in which the polymer and/or bioactive agent are dispersed so as to form a slurry or a dispersion.

Solvents of the present disclosure can include one or more hydrophobic solvents. Examples of suitable hydrophobic solvents include non-polar solvents including one or more C₄-C₁₂ straight, branched, or cyclic alkanes, dimethyl formamide, dimethyl sulfoxide, tetrahydrofuran, chloroform, acetone, acetonitrile, dioxane, dimethyl acetamide, N-methyl pyrrolidone, or combinations thereof. Useful solvents for solution coating include, for example, n-pentane, n-hexane, isooctane, n-heptane; n-octane; n-nonane, and combinations thereof.

The solvent may also include a blend of solvents that are a mixture of one or more hydrophobic solvents as provided herein, with one or more non-aqueous polar solvents. Suitable non-aqueous polar solvents can include alcohols, esters (such as acetates), organic acids (such as acetic acid), and glycols. Examples include isopropanol, propanol, diethylene glycol, propylene glycol, ethylene glycol, N-methyl pyrrolidone, glycerol, combinations of these, and the like. By utilizing a solvent system of this nature, the films may dry more rapidly. Moreover, a minimal amount of the polar solvent is used in order to increase the solubility of polymer systems with hydrophilic and charged monomers.

In various embodiments, for polymers of the present disclosure having the hydrophobic components and the charged components, the solvent can include about 85 to about 100 weight percent of one or more non-polar solvents (e.g., alkanes) and about 0 to about 15 weight percent of one or more non-aqueous polar solvents. In one embodiment, the solvent includes about 90 to about 100 percent by volume of one or more non-polar solvents selected from the group consisting of pentane, hexane, isooctane, or mixtures thereof, and about 0 to about 10 percent by volume of one or more non-aqueous polar solvent selected from the group consisting of an alcohol (e.g., isopropanol), ethyl acetate, acetone, or mixtures thereof. In an additional embodiment, the solvent includes about 90 to 99 percent by volume of one or more non-polar solvents selected from the group consisting of pentane, hexane, isooctane or mixtures thereof, and about 1 to about 10 percent by volume of one or more non-aqueous polar solvent selected from the group consisting of an alcohol (e.g., isopropanol), ethyl acetate, acetone, or mixtures thereof. In a further embodiment, the solvent includes about 95 to 99 percent by volume of one or more non-polar solvents and about 1 to 5 percent by volume of one or more non-aqueous polar solvents. In some embodiments, the non-aqueous polar solvent is isopropanol, and the alkane is isooctane.

For polymers of the present disclosure having the hydrophobic components, the charged components and the hydrophilic components, the solvent can include about 60 to 85 percent by volume of water and about 15 to 40 percent by volume of ethanol. In an additional embodiment, the solvent includes about 65 to 75 percent by volume of water and about 25 to 35 percent by volume of ethanol. In a further embodiment, the solvent includes about 70 percent by volume of water and about 30 percent by volume of ethanol.

Additional compounds can be added to the solvent to enhance and/or increase the solubility of the polymer in the solvent. For example, for various embodiments of the polymers provided herein, solubility in an aqueous system can be improved by ionizing carboxylic acid groups on the polymer through the use of a volatile base compound. Examples of suitable base compounds for providing this ionization include, but are not limited to, ammonium hydroxide, 2-aminomethylpropanol (2-AMP), and mixtures thereof, which ionization can help to increase the solubility of the polymer. After application of the film forming composition, the solvent and the volatile base compound evaporate resulting in protonation of the carboxylic acid group on the polymer. The acid groups on the polymer can then strongly associate, giving strength to the resulting film and providing for greater wash resistance of the film. Other substances may be added to the solvent and/or the film forming composition for plasticization, improved adhesion, and/or rheology control, and the like.

The bioactive agent of the present disclosure may be incorporated into the film forming composition for ready or continual release to the biological surfaces to which the film is applied. As provided herein, the bioactive agents that are hydrophilic remain miscible with the polymer of the present disclosure in the liquid state and the film state. The bioactive agent can include, but is not limited to, a therapeutic agent that can be released to the underlying biological surface of the film and/or a cosmetic agent provided for aesthetic purposes. As used herein, a “therapeutic agent” is one that produces a local or systemic effect in a subject and is used to encompass substances intended for use in the diagnosis, cure, mitigation, treatment, or prevention of disease and/or in the enhancement of desirable physical development and conditions in a subject. The term “subject” used herein is taken to include mammals (e.g., humans) and non-mammals.

Examples of the therapeutic agent include, but are not limited to, therapeutically effective amounts of an antimicrobial, topical anesthetics, antifungals, antioxidants, and mixtures thereof. Additional examples of the therapeutic agent include synthetic or naturally occurring agents, and include, without limitation, pharmacologically active polypeptides (which is used herein to encompass a polymer of L- or D-amino acids of any length including peptides, oligopeptides, proteins, enzymes, hormones, etc.), saccharides (e.g., mono-, di-, poly-saccharides, and mucopolysaccharides), vitamins, and the like. Examples of cosmetic agents include, but are not limited to, pigments, dyes, and combinations thereof. Other types of bioactive agents which may be desirable to incorporate include UV and IR absorbers.

According to the present disclosure, the bioactive agent of the present disclosure is a hydrophilic material selected from the group consisting of the therapeutic agent, the cosmetic agent, and mixtures thereof. Suitable examples of bioactive agents can include antimicrobial agents such as antibiotics such as ciprofloxacin, norfloxacin, clofoctol, and the like, and antifungal and antiseptic agents such as chlorhexidine, sanguinarine extract, metronidazole, quaternary ammonium compounds, such as cetylpyridinium chloride and benzalkonium chloride (BZK), and the like; anti-inflammatory agents such as ibuprofen, fenbufen, cortisone, and the like; vitamins such as vitamins A, C and E; anesthetic agents such as benoxinate, benzocaine, lidocaine, procaine, and the like; antihistamines; antipyretics; biocides; bactericides; biomolecules such as bacteriocins, antibodies, enzymes, and the like; nonsteroidal anti-inflammatory drugs; proteinaceous materials, such as collagen; preservatives; opacifying agents; coloring agents such as pigments, dyes, and the like; pH-adjusting agents; surfactants, such as anionic, nonionic, cationic and zwitterionic or amphoteric surfactants; and buffers and salts to buffer the pH and ionic strength of the film forming composition.

Additives that can be combined with the film forming composition can include, but are not limited to, viscosity and flow control agents to adjust the viscosity and thixotropy of the mixture to a desired level, antioxidants to improve oxidative stability of the film, surfactants, emulsifiers, preservatives, stabilizers, diluents, and air release agents or defoamers, plasticizers, adhesion promoters, or combination of these, for example.

In one embodiment, each bioactive agent is present within (i.e., incorporated within) the film forming composition in an amount of at least about 0.1 weight percent (wt %), based on the total weight of the film forming composition and the bioactive agents. In an additional embodiment, the bioactive agent is present within the film forming composition in an amount of at least about 1 wt %, based on the total weight of the film forming composition and the bioactive agents. In a further embodiment, the bioactive agent is present within the film forming composition in an amount of at least about 5 wt %, based on the total weight of the film forming composition and the bioactive agents. Typically, the amount of each bioactive agent will be at or below its solubility limit in the film forming composition.

Film forming compositions of the present disclosure could also be used for applications such as sunscreens with the incorporation of UV absorbers. Still other uses include forming films for use in eliminating chapped lips, treating skin, and providing protection to skin and other surfaces which may be medicated prior to application of the film.

In some embodiments, films formed from the film forming composition can have a thickness of the film that is no greater than about 1000 microns, no greater than about 500 microns, and/or no greater than about 100 microns. A film can be as thin as desired (e.g., 1 nanometer or thinner), but are typically not thinner than about 10 nanometers to about 100 nanometers. Generally, the film thickness is limited by the process used to form the film. For the embodiments herein, the thickness of the film does not have to be constant or uniform. Furthermore, the thickness of the film can be used to tune the duration of time over which the biological agent is released.

Film forming compositions of the present disclosure can further be included in a kit. The kit can include a quantity of the film forming composition of the present disclosure provided in a dispensing container. In one embodiment, the dispensing container includes a bottle having an applicator that releasably attaches to the bottle. The kit can be provided in a package having instructions for use of the dispensing container and the film forming composition. Examples of a suitable applicator include, but are not limited to a brush, a rod, a sponge, a cloth, a dropper, a pump sprayer, or mist forming structure or any other usable technique or structure for applying a liquid to a surface.

The invention has been described with reference to various specific and preferred embodiments and will be further described by reference to the following detailed examples. It is understood, however, that there are many extensions, variations, and modifications on the basic theme of the present invention beyond that shown in the examples and detailed description, which are within the spirit and scope of the present invention.

### EXAMPLES

A number of embodiments of the invention are illustrated by the following examples. However, the particular materials and amounts thereof recited in these examples, as well as other conditions and details, are to be interpreted to apply broadly in the art and should not be construed to unduly restrict or limit the invention in any way.

All reactions for Examples 3, 6 and 7 were performed in oven-dried glassware under a positive pressure of argon or nitrogen gas. The air- and moisture-sensitive solutions were transferred by means of a syringe into rubber-septum-capped reaction vessels. Reaction mixtures and chromatographically collected fractions were concentrated on a rotary evaporator (ca. 20° C./20 torr). Commercial grade reagents were used without further purification, except for monomers, which were degassed by applying a nitrogen- or argon-stream for 30 min. Monomer inhibitors were removed by distillation or filtration over Aldrich-inhibitor-remover [9003-70-07]. Polymerization mixtures were carried out and sealed in a glove box under a nitrogen or argon atmosphere, and performed at temperatures indicated herein. Size Exclusion Chromatography (SEC) was performed using automated gel permeation chromatography (GPC) systems and different sets of columns and eluents depending on the polarity of the polymers. In general, SEC was performed in accord with U.S. Pat. Nos. 6,296,771, 6,294,338, 6,265,226, 6,260,407 and 6,175,409, each of which is incorporated herein by reference. Molecular weight and polydispersity index (PDI) are referred to linear polystyrene standards. Monomer conversion was determined by ¹H-NMR on a Brucker AC 400 (400 MHz) or by GC on a HP-6890 automated system. Glass transition temperatures for the polymers were measured using a Parallel DMTA Workflow (Symyx Technologies, Santa Clara, Calif.).

In the present examples, the following abbreviations are used: “EHMA” is 2-ethyl hexyl methacrylate, “MMA” is methyl methacrylate, “MAA” is methacrylic acid, and “MEMA” is 2-methoxyethyl methacrylate. Unless otherwise noted, reagents were purchased from Sigma-Aldrich of Milwaukee, Wis. 2-Ethyl hexyl methacrylate (EHMA) was purchased from Alfa Aesar of Ward Hill, Mass. The water used in all experiments was distilled and degassed prior to use.

### Example 1

Library Studio® software (Symyx Technologies, Inc., Santa Clara, Calif., USA) was used to design a monomer library for the random copolymerization of MAA, EHMA and MMA. Polymerization reactions were prepared by an emulsion process using a Parallel Polymerization Reactor® (Symyx Technologies, Inc., Santa Clara, Calif., USA) from the recipe information supplied by the Library Studio® software. Stock solutions for the monomers are as provided below in Table I.

Twenty-four reaction vessels of the Parallel Polymerization Reactor® were configured as follows: an array of twenty-four sealed stainless steel reactor chambers, each equipped with speed-controlled rotary shaft stirrer paddles, disposable glass liner reaction vessels with volume capacity of about 8 ml, nitrogen gas manifold inlet and outlet, thermostatically controlled heating, and inlet lines into each of the reactor vessels, supplied by pump and valve distribution systems. The feed lines of each distribution system were primed with the corresponding stock solutions described below (Table I). The reactor was assembled in a clean, empty state, and sealed. The reactor was flushed 5 times, where each flush cycle includes pressurizing with nitrogen to a pressure of 60 psig, followed by venting to flush air from the system. Finally, the system was maintained under an ambient (1 atm) nitrogen atmosphere during the course of the reaction.

TABLE I
Stock Solutions from Example 1

| Feed | Component | Composition |
|---|---|---|
| 1 | 2-Ethyl Hexyl | EHMA, 100% |
| | Methacrylate | |
| | (EHMA) | |
| 2 | Ascorbic acid | 2.0 wt % in water |
| | (Asc) | |
| 3 | tert-Butyl Peroxide | 1.5 wt % in water |
| | (t-BHP) | |
| 4 | Methacrylic Acid | 2.2 wt % in water |
| | (MAA) | |
| 5 | Methyl | 55 wt % in EHMA |
| | Methacrylate | |
| | (MMA) | |
| 6 | Water | Water, 100% |

For the array of twenty-four reaction vessels (four rows by six columns) the percent of total monomers for MAA was 2.76% for row one, 1.84% for row two, 0.92% for row three and 0.0% for row four, while the theoretical molar ratio of total monomers for EHMA/MMA was 55/45 for column 1, 58.1/41.9 for column 2, 61.6/38.4 for column 3, 65/35 for column 4, 70/30 for column 5, and 75/25 for column 6 of the array.

The addition protocol was as follows. In a first stage, the surfactant solution was added followed by the addition of the solvent. After 45 minutes to 1 hour, 10% of EHMA, and 9% of MMA and MAA stock solutions were added and the temperature was set to 58° C. and stirring was begun. After 20 minutes, 30% of the Asc solution and 5% of the t-BHP was added to each reaction vessel. The second stage began after 20 minutes, during which most of the remaining monomers (90% of EHMA, 90% MMA and 90% MAA) and initiators were added semicontinuously in 100 steps during 9 hours. The remaining 1% of MMA and MAA was then added in a single step. Heating was maintained for 1 hour at 58° C. The samples where then filtered over glasswool and directly precipitated in acetone. Solids were then washed several times with water and dried overnight.

### Example 2

The polymers synthesized in Example 1 were then subjected to a screening methodology to investigate various performance properties, including flexural durability, elongational durability, and wash resistance. The wash resistance test provided information on solubility of the films formed with polymers from Example 1 in soap water solutions and the thickness increase of non-soluble polymers due to water absorption. The objective of this test was to assess whether a polymer film dissolves in, or absorbs, water under conditions similar to those presented during personal hygiene or during household chore activities such as dishwashing. Polymer films were formed by dispensing 20 μl of a 20% polymer solution on a Kapton® (DuPont, Wilmington Del.) covered aluminum plate. After the films were dried by leaving them on a hot stage set at 37° C., the thickness (H1) of the films was read and recorded using a scanning laser profilometer (Symyx Technologies, Santa Clara, Calif.).

The effect of water on film integrity was assessed by immersing the plate (face down) in a detergent solution at 43° C. for 15 minutes. This solution was contained in a Petri dish in which gentle magnetic stirring was applied to homogenize the solution temperature and to apply some shear to the films. After the required time, the plate was washed with clean water and the excess water was eliminated by a gentle blow of compressed air. The thickness of the films (H2) was then measured immediately while the films were still wet. A decrease in thickness reflected partial dissolution of the polymer. An increase in thickness indicated swelling of the film. Samples that did not show changes in thickness were deemed wash resistant polymers. Intermediate cases (simultaneous partial dissolution and swelling of the film) will also result in thickness close to the original values, and may mislead the interpretation of the results. To clear any doubts arising from the wet thickness reading, a third reading (H3) was performed after the films were dried a second time. The ratio H3/H1 quantifies the extent to which the material resists dissolution, i.e. wash resistance; the ratio H2/H3 measures the degree of swelling upon immersion in the wash liquid.

The results of wash resistance of the films formed with the polymers from Example 1 are shown in Table II. All polymers exhibit good water resistance with all thickness changes within 2% (H3/H1).

TABLE II
Water Resistance Data of the Polymers from Example 1

| Polymer | H3/H1 | H3/H2 | Polymer | | |
|---|---|---|---|---|---|
| Well # | Wash resistance | swelling | Well # | H3/H1 | H3/H2 |
| A1 | 1.00 | 0.99 | C1 | 1.00 | 1.00 |
| A2 | 1.01 | 1.00 | C2 | 1.00 | 1.01 |
| A3 | 1.05 | 1.01 | C3 | 0.98 | 0.95 |
| A4 | 1.01 | 0.93 | C4 | 1.00 | 0.99 |
| A5 | 1.01 | 1.04 | C5 | 1.00 | 0.99 |
| A6 | 1.00 | 1.00 | C6 | 1.00 | 0.99 |
| B1 | 0.99 | 1.02 | D1 | 0.98 | 0.99 |
| B2 | 1.00 | 1.01 | D2 | 0.99 | 0.99 |
| B3 | 1.01 | 1.00 | D3 | 0.99 | 0.99 |
| B4 | 1.02 | 1.02 | D4 | 0.98 | 0.99 |
| B5 | 0.99 | 0.99 | D5 | 0.98 | 0.99 |
| B6 | 1.00 | 1.00 | D6 | 1.00 | 1.04 |

In the flexural durability test, a film of each of the polymers was formed on a Viton® sheet (DuPont, Wilmington Del.). The Viton surface was first prepared by bead blasting to obtain a rougher surface and improve the adhesion of the resulting polymer film. The film was prepared by dispensing 40 μl of a 15 wt % polymer solution in Chloroform on the elastic substrate and allowing it to spread to cover approximately 1 cm². The films were left to dry overnight on top of a hot stage at 37° C. The dry thickness of the polymer film was approximately 20 to 30 μm.

The films were then placed in an ambient controller chamber (35° C. at 50% relative humidity) for 6 hours for equilibration. The flexural test was carried out by rolling the elastic substrate with the film around a sharp angled pole so that every section of the film was bent at 90 degrees sometime during the experiment. This movement was repeated ten times (strokes). Any cracking or wrinkling of the polymer film was captured in images using a microscope. No changes were observed in the polymer films after 10 strokes.

For the elongational durability test, polymer films were prepared on bead blasted Viton® strips in the same way as in the flexural durability test. Once the films were dried and equilibrated as discussed above, the strips were subjected to elongation under mechanical and ambient controlled conditions (35° C. at 50% relative humidity). The strips were subjected to elongation to 100% stain from their original length (40 mm) (i.e., the films were stretched to twice their original length) at an elongation rate of 900%/minute. Once the elongation reached the maximum, results (cracking, delamination, etc.) were recorded. After the films were relaxed to the original length, pictures were taken with the microscope to record any structural change of the film after relaxation. The film of polymer A4 shows very slight edge delamination, and the film of polymer B6 shows some cracks after relaxation. No structure changes were observed for the rest of films.

### Example 3

A polymer formed from monomers of EHMA, MMA, and MAA was prepared, characterized and tested for solubility in mixtures of one or more hydrophobic solvents and one or more non-aqueous polar solvents. Molar percentages of the monomers EHMA, MMA and MAA for the reaction were 54 mol. %, 44.16 mol. %, and 1.84 mol. %, respectively. The polymer was prepared using a redox initiating system (peroxide initiator) at 0.3 weight percent based on total weight of the monomer in an emulsion free-radical polymerization process. The total polymerization reaction volume was one liter.

A 2 L, three-neck, round-bottom flask was equipped with mechanical stirring, a thermometer and adapters, heating mantle, and a flow control to provide an argon atmosphere and inlet (a septum) to lines from individual stock solutions (Table III). A surfactant mixture of 6.102 g of Tergitol 15S5 (Dow Chemical Company), 2.029 g of Tergitol 15S40 (Dow Chemical Company), 0.001 g of sodium acetate (Sigma Chemical), and 6.102 g of Dowfax 2A1 (Dow Chemical Company) was introduced into the flask. The flask was purged with argon for 30 minutes. Water (367.97 mL) was introduced in the flask via double-tip syringe needle on the surfactant mixture. The emulsion mixture was then stirred for 15 to 30 minutes. The emulsion mixture in the flask was then heated to a constant temperature of 58° C.

TABLE III
Preparation of the Stock Solutions for EHMA/MAA/MMA polymer of
Example 3. Stock solutions and water were degassed by the inert
gas bubbling technique (Ar) for about 30 minutes to 1 hour.

| | | Stock Solution | |
|---|---|---|---|
| Name | Components | Composition | |
| 2-Ethyl Hexyl | EHMA | 153.654 | ml |
| Methacrylate | | | |
| (EHMA) | | | |
| Ascorbic acid (Asc) | Asc | 2.041 | g |
| | Water | 100 | ml |
| tert-Butyl Peroxide (t- | t-BHP | 1.516 | g |
| BHP) | Water | 100 | ml |
| Methacrylic Acid | MAA | 3.599 | g |
| (MAA) | Water | 160 | ml |
| Methyl Methacrylate | MMA | 103.889 | g |
| (MMA) | EHMA | 85 | g |
| Water | Water | 367.97 | ml |

An initial charge of monomers of the MMA, EHMA and MAA stock solutions according to Table IV, below, was introduced into the flask. The emulsion mixture was left to equilibrate under stirring for 20 minutes at a constant temperature of 58° C. After 20 minutes, an initial charge of the Asc stock solution (Table IV) was added to the flask. An initial charge of the t-BHP stock solution (Table IV) was then added after an additional 10 minutes to provide the free-radical initiator. After an additional 20 minutes, a remaining volume of the free-radical initiator (Asc/t-BHP) and the MMA, EHMA and MAA stock solutions were fed to the flask in 100 equal steps over a 9 hour interval, as indicated in Table IV. A late charge of monomers of the MMA stock solution and the MAA stock solution according to Table IV was then added in the emulsion mixture in a single step after the 9 hour reaction time. The resulting emulsion mixture was then left under stirring at 58° C. for 1 hour before being cooled to room temperature.

TABLE IV
Dispense of the Stock Solutions of Table III - (*)volumes
dispensed in 100 steps over 9 hours, (**)addition in one
step after the 9 hour reaction time.

| Stock | | | | | |
|---|---|---|---|---|---|
| Solution | Total | | | | Late |
| (Table | Volume | Initial | Initial | Volume to | Charge |
| III) | (uL) | Charge | Charge (uL) | dispense (uL)(*) | (uL)(**) |
| MMA | 169076 | 9% | 15216.84 | 152168.4 | 1690.76 |
| EHMA | 153654 | 10% | 15365.4 | 138288.6 | — |
| MAA | 138090 | 9% | 12428.1 | 124281 | 1380.90 |
| Asc | 76573 | 30% | 22971.9 | 53601.1 | — |
| t-BHP | 83019 | 10% | 8301.9 | 74717.1 | — |

The resulting emulsion mixture was then passed through glasswool to remove any solids. The emulsion mixture containing the polymer was precipitated by a dropwise addition of the mixture into methanol or acetone at room temperature. The resulting polymer was then filtered and thoroughly washed with water on a Büchner funnel until no foam was observed through the filter. The polymer was then freeze dried to remove adsorbed water. A further purification of the polymer was performed by a reverse precipitation technique during which the polymer was dissolved into the minimum amount of solvent and the non-solvent can be added dropwise to the solution. This process allows for removal of small molecules (unreacted monomers, initiators, surfactants or small polymeric chains).

During the process, the polymer (300 g) was then divided into two lots of 150 g each (for convenience only) and dissolved in 500 mL of a mixture isopropanol-isooctane (50/50 vol/vol) in a beaker. Ethanol (4 L) was then added dropwise to the mixture of the polymer and the isopropanol-isooctane that resulted in a cloudy mixture. The cloudy mixture was left under stirring for 1 hour and then placed in a refrigerator (3° C.) overnight. A resulting supernatant was then discarded and the polymer collected as a gummy material at the bottom of the beaker. The polymer was then washed several times with ethanol, and then transferred to a plastic container. The polymer was then dried at 85° C. for 16 hours before analysis.

The polymer was then characterized by H¹ NMR and gel permeation chromatography (GPC). The molar ratio of EHMA/MMA units in the polymer was determined by ¹H NMR to be 53 mol % EHMA, and 47 mol % MMA. The molecular weight obtained by GPC showed a broad peak with a weight average molecular weight (Mw) of 495,000 g/mol and a polydispersity index (PDI) of 2.35. The glass transition temperature (T_{g}) for the EHMA/MAA/MMA polymer was determined to be Tg=42.0° C. using a Parallel DMTA Workflow (Symyx Technologies, Santa Clara, Calif.).

### Example 4

The polymer 0.15 g from Example 3 was mixed with 1 ml of isooctane containing 5 wt % isopropanol to make a 15 wt % solution of the polymer. Polymer films were prepared from the 15 wt % solution of the polymer in the same way as in Example 2 for water resistance test, and for durability test. The thickness change of the polymer film was less than 2% after the film was washed in hot soap water and re-dried for the thickness measurement. No changes to the film surface were observed during the flexural and elongational durability tests.

### Example 5

The polymer 0.15 g from Example 3 was mixed with 1 ml of isooctane containing 5 wt % isopropanol and 0.13 wt % of Benzalkonium Chloride (Aldrich) to make a 15 wt % solution of the polymer. Polymer films were prepared from the 15 wt % solution of the polymer in the same way as in Example 2 for water resistance test, and for durability test. The thickness change of the polymer film was less than 2% after the film was washed in hot soap water and re-dried for the thickness measurement. No changes to the film surface were observed during the flexural and elongational durability tests.

### Example 6

A polymer formed from monomers of EHMA, MMA, MAA, and MEMA was prepared, characterized, and tested for solubility in mixtures of one or more hydrophobic solvents and one or more non-aqueous polar solvents. Molar percentages of the monomers EHMA, MMA, MEMA, and MAA for the reaction were 49 mol. %, 39 mol. %, 11 mol. %, and 1 mol. %, respectively. The polymer was prepared as a free-radical polymerization process using a thermal initiating system (peroxide initiator) at 0.8 mole % based on total moles of the monomer in isooctane solution at 80° C. The total polymerization reaction volume was 0.4 liter.

A 1 L three-neck round bottom flask was prepared with mechanical stirring, a thermometer and adapters, and a flow control to provide inert atmosphere and inlet (a septum) to lines from individual stock solutions (Table V). The flask was also provided with a heater.

TABLE V
Preparation of the Stock Solutions for the EHMA/MMA/MEMA/MAA
polymer of Example 6. Stock solutions were degassed by the inert
gas bubbling technique (Ar) for about 30 minutes.

| | | |
|---|---|---|
| | | Stock Solution |
| Name | Components | Composition |

| | | | |
|---|---|---|---|
| 2-Ethyl Hexyl | EHMA | 167.528 | ml |
| Methacrylate | Isooctane | 4.398 | ml |
| (EHMA) | | | |
| Methyl Methacrylate | MMA | 63.654 | ml |
| (MMA) | Isooctane | 2.678 | ml |
| 2-methoxyethyl | MEMA | 24.369 | ml |
| methacrylate | Isooctane | 4.347 | ml |
| (MEMA) | | | |
| Methacrylic Acid | Methacrylic acid | 1.294 | ml |
| (MAA) | Isooctane | 26.588 | ml |
| Lauroyl peroxide | Lauroyl peroxide | 4.866 | g |
| | Isooctane | 100.089 | ml |
| Isooctane | Isooctane | 0.189 | ml |

An initial charge of monomers of the EHMA, MMA, MEMA, and MAA stock solutions, the solvent and an initiator (lauroyl peroxide) according to Table VI, below, were introduced into the flask and stirred. The mixture was then heated to a constant temperature of 80° C. After 10 minutes, a feed of the monomer stock solutions was added in 100 steps and a feed of the initiator was added in 10 steps over a 5 hour interval. After the reagents were added, the mixture was left under stirring at 80° C. for 1 more hour. Before cooling, the mixture was diluted with 200 ml of isopropanol. Once at room temperature, the polymer was precipitated by a slow addition of ethanol (up to 3 liters). The precipitate was washed several times with ethanol then re-dissolved in isooctane/isopropanol 50/50 vol % and the precipitation with ethanol was repeated. After several washes with ethanol the precipitate was placed in a plastic flask and dried under vacuum at a temperature of 65° C. for 48 hours.

TABLE VI
Dispense of the Stock Solutions of Table V - (*) 100 steps
in 5 hours, (**) 10 steps in 5 hours.

| Stock | Total | | Initial | |
|---|---|---|---|---|
| Solution | Volume | Initial | Charge | Volume to |
| (Table V) | (mL) | Charge | (mL) | dispense (mL)(*) |
| MMA | 66.33 | 10% | 6.63 | 59.70 |
| EHMA | 171.93 | 10% | 17.19 | 154.74 |
| MEMA | 28.72 | 10% | 2.87 | 25.85 |
| MAA | 27.88 | 10% | 2.79 | 25.09 |
| Lauroyl | 104.95 | 35% | 36.73 | 94.45(**) |
| peroxide | | | | |

The EHMA/MMA/MEMA/MAA polymer was then characterized by H¹ NMR and gel permeation chromatography (GPC). The molar ratio of the incorporated EHMA, MMA, and MEMA units was determined by ¹H NMR to be 48 mol. % EHMA, 41 mol. % MMA, and 11 mol. % MEMA. Analysis of the polymer with GPC showed a broad peak with a weight-average molecular weight (Mw) of 129,000 g/mol and polydispersity index (Mw/Mn) of 1.90. The glass transition temperature for the EHMA/MMA/MEMA/MAA polymer was found to be Tg=41.2° C.

### Example 7

A polymer formed from monomers of EHMA, MMA, MAA, and MEMA was prepared, characterized and tested for solubility in mixtures of one or more hydrophobic solvents and one or more non-aqueous polar solvents. Molar percentages of the monomers EHMA, MMA, MEMA, and MAA for the reaction were 60 mol. %, 32 mol. %, 7 mol. %, and 1 mol. %, respectively. The polymer was prepared as a free-radical polymerization process using a thermal initiating system (peroxide initiator) at 0.8 mole percent based on total moles of the monomers in isooctane solution at 80° C. The total polymerization reaction volume was 0.4 liter.

A 1 L three-neck round bottom flask was prepared with a mechanical stirring, a thermometer and adapters: a flow control to provide inert atmosphere and inlet (a septum) to lines from individual stock solutions (Table VII). The flask was also provided with a heater.

TABLE VII
Preparation of the Stock Solutions for the EHMA/MMA/MEMA/MAA
polymer of Example 7. Stock solutions were degassed by the inert
gas bubbling technique (Ar) for about 30 minutes.

| | | Stock Solution | |
|---|---|---|---|
| Name | Components | Composition | |
| 2-Ethyl Hexyl | EHMA | 192.656 | ml |
| Methacrylate | Isooctane | 7.665 | ml |
| (EHMA) | | | |
| Methyl Methacrylate | MMA | 49.051 | ml |
| (MMA) | Isooctane | 2.064 | ml |
| 2-methoxyethyl | MEMA | 14.564 | ml |
| methacrylate | Isooctane | 9.009 | ml |
| (MEMA) | | | |
| Methacrylic Acid | MAA | 1.215 | ml |
| (MAA) | Isooctane | 24.971 | ml |
| Lauroyl peroxide | Lauroyl peroxide | 4.57 | g |
| | Isooctane | 94 | ml |
| Isooctane | Isooctane | 0.236 | ml |

An initial charge of monomers of the EHMA, MMA, MEMA, and MAA stock solutions, isooctane, the lauroyl peroxide (initiator), as indicated in Table VIII, were added to the flask. The mixture was heated to a constant temperature of 80° C. After 10 minutes, a feed of the monomer stock solutions was added in 100 steps and a feed of the initiator was added in 10 steps over a 5 hour interval. After the reagents were added, the mixture was left under stirring at 80° C. for 1 more hour. Before cooling, the mixture was diluted with 200 ml of isopropanol. Once at room temperature, the polymer was precipitated by a slow addition of ethanol (up to 3 liters). The precipitate was washed several times with ethanol then re-dissolved in isooctane/isopropanol 50/50% volume and the precipitation with ethanol was repeated. After several washes with ethanol the precipitate was placed in a plastic flask and dried under vacuum at 65° C. for 48 hours.

TABLE VIII
Dispense of the Stock Solutions - (*) 100 steps in 5 hours,
(**) 10 steps in 5 hours.

| Stock | | | | |
|---|---|---|---|---|
| Solution | Total | | Initial | |
| (Table | Volume | Initial | Charge | Volume to |
| VII) | (mL) | Charge | (mL) | dispense (mL)(*) |
| MMA | 51.11 | 10% | 5.11 | 46.0 |
| EHMA | 200.32 | 10% | 20.03 | 180.29 |
| MEMA | 23.57 | 10% | 2.36 | 21.21 |
| MAA | 26.19 | 10% | 2.62 | 23.57 |
| Lauroyl | 98.57 | 35% | 34.50 | 64.07(**) |
| peroxide | | | | |

The polymer was then characterized by H¹ NMR and gel permeation chromatography (GPC). The molar ratio of the incorporated EHMA, MMA, and MEMA units contents in the polymer were determined by ¹H NMR to be 60 mol. % EHMA, 33 mol. % MMA, and 7 mol. % MEMA. Analysis of the polymer with GPC showed a broad peak with a weight-average molecular weight (Mw) of 182,000 g/mol and polydispersity index (Mw/Mn) of 2.08. The glass transition temperature for the polymer was found to be Tg=32.0° C.

### Example 8

The following additional polymer libraries were synthesized and screened as described herein.

A) MAA (2 to 0.5 mol %) and EHMA/MMA (from 50/50 to 62/37 mole ratio).

B) MAA (1 mol %), MMA (from 32 to 42 mol %), MEMA (from 1 to 11 mol %), EHMA (remaining mol %).

All reactions were done on an 8 mL scale. For polymer library A, water was used as the solvent. For polymer library B, isooctane was used as the solvent. The total monomeric concentration for both reactions was 10 wt %. The amount of initiator used was 1 wt % compared to the total monomer in the mixture. The hydrophobic and charged monomer stock solution has a concentration of 15 wt % in the solvent.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A film forming composition, comprising:
a polymer comprising from about 80 mole percent to about 100 mole percent of a hydrophobic component, and from about 0 to about 20 mole percent of a charged component;
a bioactive agent; and
a solvent in which the polymer and the bioactive agent are homogeneously dispersed in the composition.

2. The film forming composition of claim 1, where the polymer has a structure that is selected from the group consisting of random polymers, block polymers, graft polymers and hyperbranched polymers.

3. The film forming composition of claim 1, where the hydrophobic component is a material comprising a polymerizable monomer, oligomer, or polymer.

4. The film forming composition of claim 3, where the material for the hydrophobic component is selected from the group consisting of methyl acrylate (MA), methyl methacrylate (MMA), ethyl acrylate (EA), ethyl methacrylate (EMA), 2-methoxyethyl acrylate (MEA), 2-methoxyethyl methacrylate (MEMA), styrene (Sty), vinyl acetate (VA), vinyl neodecanoate (VND), 2-ethyl hexyl methacrylate (EHMA), 2-ethylhexyl acrylate (EHA), butyl acrylate (BA), butyl methacrylate (BMA), lauryl acrylate (LA), and mixtures thereof.

5. The film forming composition of claim 1, where the charged component is a material comprising a polymerizable monomer, oligomer, or polymer.

6. The film forming composition of claim 5, where the material for the charged component is selected from the group consisting of N,N-dimethylaminoalkyl acrylate, N,N-dimethylaminoalkyl methacrylate, N,N-dimethylaminoethyl acrylate (DMAEA), [2(methacryloyloxy)ethyl]trialkylammonium salt, [2-(acryloyloxy)ethyl]trialkylammonium salt, [2-(acryloyloxy)ethyl]trimethylammonium chloride (AETMAC), N,N-dimethylaminoalkyl methacrylamide, N,N-dimethylaminoalkyl acrylamide, N,N,N-trimethylaminoalkyl methacrylamide salt, N,N,N-trimethylaminoalkyl acrylamide salt, (vinylbenzyl)trialklyammonium salt, (vinylbenzyl)trimethylammonium chloride (VBTMAC), acrylic acid (AA), methacrylic acid (MAA), itoconic acid (IC), crotonic acid, maleic anhydride, maleic acid, fumaric acid, mono-2-(methacryloyl)ethyl succinate (MAES), 2-acrylamido-2-methyl-1-propanesulfonic acid (AMPS), vinylphosphonic acid (VPA), and mixtures thereof.

7. The film forming composition of claim 1, where the polymer comprises about 5 mole percent or less of methacrylic acid (MAA), with the remaining mole percent of the polymer being 2-ethyl hexyl methacrylate (EHMA) and methyl methacrylate (MMA) in a ratio of about 75/25 to about 50/50 mole percent EHMA/MMA.

8. The film forming composition of claim 7, where the polymer comprises about 2 mole percent or less of methacrylic acid (MAA), with the remaining mole percent of the polymer being 2-ethyl hexyl methacrylate (EHMA) and methyl methacrylate (MMA) in a ratio of about 55/45 to about 50/50 mole percent EHMA/MMA.

9. The film forming composition of claim 8, where the solvent comprises about 90 to about 100 percent by volume of octane and about 0 to about 10 percent by volume of isopropanol.

10. The film forming composition of claim 1, where the polymer comprises about 0 to about 5 mole percent of methacrylic acid (MAA), about 30 to about 45 mole percent of methyl methacrylate (MMA), about 1 to about 15 mole percent of 2-methoxyethyl methacrylate (MEMA), with the remaining mole percent of the polymer being 2-ethyl hexyl methacrylate (EHMA).

11. The film forming composition of claim 10, where the polymer comprises about 0 to 2 mole percent of methacrylic acid (MAA), about 35 to about 40 mole percent of methyl methacrylate (MMA), about 5 to 15 mole percent of 2-methoxyethyl methacrylate (MEMA), with the remaining mole percent of the polymer being 2-ethyl hexyl methacrylate (EHMA).

12. The film forming composition of claim 11, where the solvent comprises about 85 to 100 percent by volume of octane and about 0 to 15 percent by volume of isopropanol.

13. The film forming composition of claim 1, where the polymer further comprises a hydrophilic component, and the polymer comprises about 0 to about 10 mole percent of the charged component, the remaining mole percent of the polymer being the hydrophilic component and the hydrophobic component in a ratio of about 60/40 percent to about 80/20 percent.

14. The film forming composition of claim 13, where the hydrophilic component is a material comprising a polymerizable monomer, oligomer, or polymer.

15. The film forming composition of claim 14, where the material for the hydrophilic component is selected from the group consisting of N-vinylpyrrolidone (VPL), 2-hydroxyethyl acrylate (HEA), 2-hydroxylethyl methacrylate (HEMA), N,N-dimethyl acrylamide (DMA), N-[tris(hydroxymethyl)methyl]acrylamide (THMMAM), polyethyleneglycol acrylate (PEGA), polyethyleneglycol methacrylate (PEGMA), and mixtures thereof.

16. The film forming composition of claim 15, where the polymer comprises about 2.0 to about 7.5 mole percent of acrylic acid (AA) for the charged component, with the remaining mole percent of the polymer being methyl acrylate (MA) for the hydrophobic component, and 2-hydroxyethyl methacrylate (HEMA) for the hydrophilic component in a ratio of about 40/60 percent MA/HEMA to about 20/80 percent MA/HEMA.

17. The film forming composition of claim 16, where the solvent comprises about 60 to 85 percent by volume of water and about 15 to 40 percent by volume of ethanol.

18. The film forming composition of claim 17, where the solvent further comprises ammonium hydroxide, 2-aminomethylpropanol (2-AMP), and mixtures thereof.

19. The film forming composition of claim 1, where the bioactive agent is a hydrophilic material selected from the group consisting of a therapeutic agent, a cosmetic agent, and mixtures thereof.

20. The film forming composition of claim 19, where the therapeutic agent is selected from the group consisting of a therapeutically effective amount of an antimicrobial, a topical anesthetic, an antifungal, an antioxidant, and mixtures thereof.

21. The film forming composition of claim 19, where the cosmetic agent is selected from the group consisting of a pigment, a dye, and combination thereof.

22. The film forming composition of claim 1, where the polymer forms a water resistant film upon evaporation of the solvent.

23. The film forming composition of claim 22, where the polymer and the bioactive agent remain homogeneously dispersed upon evaporation of the solvent.

24. The film forming composition of claim 1 for use as a liquid bandage.

25. The film forming composition of claim 1, where the polymer has a solubility less than 1% by weight in deionized water at 40° C.

26. The film forming composition of claim 1, where the polymer is soluble at a concentration of at least 1% by weight in the solvent at 25° C.

27. The film forming composition of claim 26, where the solvent has a boiling point less than 75° C. at 1 atm.

28. The film forming composition of claim 27, where the solvent comprises about 90 to about 100 weight percent of one or more alkanes and about 0 to about 10 weight percent of one or more non-aqueous polar solvent.

29. The film forming composition of claim 27, where the solvent comprises about 90 to about 100 weight percent of one or more alkanes selected from the group consisting of pentane, hexane, isooctane or mixtures thereof, and about 0 to about 10 weight percent of one or more non-aqueous polar solvent selected from the group consisting of an alcohol, ethyl acetate, acetone or mixtures thereof.

30. The film forming composition of claim 28, where the non-aqueous polar solvent is isopropanol, and the alkane is isooctane.

31. A polymer for making a film forming composition, comprising:
a polymer made from a reaction product of about 80 mole percent to about 100 mole percent of a hydrophobic component, and from about 0 to about 20 mole percent of a charged component, where the polymer homogeneously disperses with a bioactive agent in a solvent.

32. The polymer of claim 31, where the polymer has a structure that is selected from the group consisting of random polymers, block polymers, graft polymers and hyperbranched polymers.

33. The polymer of claim 32, where the hydrophobic component is a material comprising a polymerizable monomer, oligomer, or polymer.

34. The polymer of claim 33, where the material for the hydrophobic component is selected from the group consisting of methyl acrylate (MA), methyl methacrylate (MMA), ethyl acrylate (EA), ethyl methacrylate (EMA), 2-methoxyethyl acrylate (MEA), 2-methoxyethyl methacrylate (MEMA), styrene (Sty), vinyl acetate (VA), vinyl neodecanoate (VND), 2-ethyl hexyl methacrylate (EHMA), 2-ethylhexyl acrylate (EHA), butyl acrylate (BA), butyl methacrylate (BMA), lauryl acrylate (LA), and mixtures thereof.

35. The polymer of claim 31, where the charged component is a material comprising a polymerizable monomer, oligomer, or polymer.

36. The polymer of claim 35, where the material for the charged component is selected from the group consisting of N,N-dimethylaminoalkyl acrylate N,N-dimethylaminoalkyl methacrylate, N,N-dimethylaminoethyl acrylate (DMAEA) [2(methacryloyloxy)ethyl]trialkylammonium salt, [2-(acryloyloxy)ethyl]trialkylammonium salt, [2-(acryloyloxy)ethyl]trimethylammonium chloride (AETMAC), N,N-dimethylaminoalkyl methacrylamide, N,N-dimethylaminoalkyl acrylamide, N,N,N-trimethylaminoalkyl methacrylamide salt, N,N,N-trimethylaminoalkyl acrylamide salt, (vinylbenzyl)trialklyammonium salt, (vinylbenzyl)trimethylammonium chloride (VBTMAC), acrylic acid (AA), methacrylic acid (MAA), itoconic acid (IC), crotonic acid, maleic anhydride, maleic acid, fumaric acid, mono-2-(methacryloyl)ethyl succinate (MAES), 2-acrylamido-2-methyl-1-propanesulfonic acid (AMPS), vinylphosphonic acid (VPA), and mixtures thereof.

37. The polymer of claim 31, where the polymer comprises about 5 mole percent or less of methacrylic acid (MAA), with the remaining mole percent of the polymer being 2-ethyl hexyl methacrylate (EHMA) and methyl methacrylate (MMA) in a ratio of about 75/25 to about 50/50 mole percent EHMA/MMA.

38. The polymer of claim 37, where the polymer comprises about 2 mole percent or less of methacrylic acid (MAA), with the remaining mole percent of the polymer being 2-ethyl hexyl methacrylate (EHMA) and methyl methacrylate (MMA) in a ratio of about 55/45 to about 50/50 mole percent EHMA/MMA.

39. The polymer of claim 38, where the solvent comprises about 90 to about 100 percent by volume of octane and about 0 to about 10 percent by volume of isopropanol.

40. The polymer of claim 31, where the polymer comprises about 0 to about 5 mole percent of methacrylic acid (MAA), about 30 to about 45 mole percent of methyl methacrylate (MMA), about 1 to about 15 mole percent of 2-methoxyethyl methacrylate (MEMA), with the remaining mole percent of the polymer being 2-ethyl hexyl methacrylate (EHMA).

41. The polymer of claim 40, where the polymer comprises about 0 to 2 mole percent of methacrylic acid (MAA), about 35 to about 40 mole percent of methyl methacrylate (MMA), about 5 to 15 mole percent of 2-methoxyethyl methacrylate (MEMA), with the remaining mole percent of the polymer being 2-ethyl hexyl methacrylate (EHMA).

42. The polymer of claim 41, where the solvent comprises about 85 to 100 percent by volume of octane and about 0 to 15 percent by volume of isopropanol.

43. The polymer of claim 31, where the polymer further comprises a hydrophilic component, and the polymer comprises about 0 to about 10 mole percent of the charged component, the remaining mole percent of the hydrophilic component and the hydrophobic component in a ratio of about 60/40 percent to about 80/20 percent.

44. The polymer of claim 43, where the hydrophilic component is a material comprising a polymerizable monomer, oligomer, or polymer.

45. The polymer of claim 44, where the material for the hydrophilic component is selected from the group consisting of N-vinylpyrrolidone (VPL), 2-hydroxyethyl acrylate (HEA), 2-hydroxylethyl methacrylate (HEMA), N,N-dimethyl acrylamide (DMA), N-[tris(hydroxymethyl)methyl]acrylamide (THMMAM), polyethyleneglycol acrylate (PEGA), polyethyleneglycol methacrylate (PEGMA), and mixtures thereof.

46. The polymer of claim 45, where the polymer comprises about 2.0 to about 7.5 mole percent of acrylic acid (AA) for the charged component, the remaining mole percent of methyl acrylate (MA) for the hydrophobic component, and 2-hydroxyethyl methacrylate (HEMA) for the hydrophilic component in a ratio of about 40/60 percent MA/HEMA to about 20/80 percent MA/HEMA.

47. The polymer of claim 46, where the solvent comprises about 60 to 85 percent by volume of water and about 15 to 40 percent by volume of ethanol.

48. The polymer of claim 47, where the solvent further comprises ammonium hydroxide, 2-aminomethylpropanol (2-AMP), and mixtures thereof.

49. The polymer of claim 31, where the bioactive agent is a hydrophilic material selected from the group consisting of a therapeutic agent, a cosmetic agent, and mixtures thereof.

50. The polymer of claim 49, where the therapeutic agent is selected from the group consisting of a therapeutically effective amount of an antimicrobial, a topical anesthetic, an antifungal, an antioxidant, and mixtures thereof.

51. The polymer of claim 50, where the cosmetic agent is selected from the group consisting of a pigment, a dye, and combination thereof.

52. The polymer of claim 31, where the polymer forms a water resistant film upon evaporation of the solvent.

53. The polymer of claim 52, where the polymer and the bioactive agent remain homogeneously dispersed upon evaporation of the solvent.

54. The polymer of claim 31 for use as a liquid bandage composition.

55. The polymer of claim 31, where the polymer has a solubility less than 1% by weight in deionized water at 40° C.

56. The polymer of claim 31, where the polymer is soluble at a concentration of at least 1% by weight in the solvent at 25° C.

57. The polymer of claim 56, where the solvent has a boiling point less than 75° C. at 1 atm.

58. The polymer of claim 57, where the solvent comprises about 90 to about 100 weight percent of one or more alkanes and about 0 to about 10 weight percent of one or more non-aqueous polar solvent.

59. The polymer of claim 58, where the solvent comprises about 90 to about 100 weight percent of one or more alkanes selected from the group consisting of pentane, hexane, isooctane or mixtures thereof, and about 0 to about 10 weight percent of one or more non-aqueous polar solvent selected from the group consisting of an alcohol, ethyl acetate, acetone or mixtures thereof.

60. The polymer of claim 59, where the non-aqueous polar solvent is isopropanol, and the alkane is isooctane.

61. A method of making a film forming composition, comprising:
dissolving
(i) a polymer formed from a reaction product of about 80 mole percent to about 100 mole percent of a hydrophobic component, and from about 0 to about 20 mole percent of a charged component, and
(ii) a bioactive agent;
in a solvent.

62. The method of claim 61, where the bioactive agent is a hydrophilic material selected from the group consisting of a therapeutic agent, a cosmetic agent, and mixtures thereof.

63. The method of claim 62, where the therapeutic agent is selected from the group consisting of a therapeutically effective amount of an antimicrobial, a topical anesthetic, an antifungal, an antioxidant, and mixtures thereof.

64. The method of claim 62, where the cosmetic agent is selected from the group consisting of a pigment, a dye, and combination thereof.

65. The method of claim 61, including evaporating the solvent to form a water resistant film of the polymer and the bioactive agent, where the polymer and the bioactive agent remain homogeneously dispersed upon evaporation of the solvent.

66. The method of claim 61, where the hydrophobic component is a material comprising a polymerizable monomer, oligomer, or polymer.

67. The method of claim 66, where the material for the hydrophobic component is selected from the group consisting of methyl acrylate (MA), methyl methacrylate (MMA), ethyl acrylate (EA), ethyl methacrylate (EMA), 2-methoxyethyl acrylate (MEA), 2-methoxyethyl methacrylate (MEMA), styrene (Sty), vinyl acetate (VA), vinyl neodecanoate (VND), 2-ethyl hexyl methacrylate (EHMA), 2-ethylhexyl acrylate (EHA), butyl acrylate (BA), butyl methacrylate (BMA), lauryl acrylate (LA), and mixtures thereof.

68. The method of claim 61, where the charged component is a material comprising a polymerizable monomer, oligomer, or polymer.

69. The method of claim 68, where the material for the charged component is selected from the group consisting of N,N-dimethylaminoalkyl acrylate N,N-dimethylaminoalkyl methacrylate, N,N-dimethylaminoethyl acrylate (DMAEA), [2(methacryloyloxy)ethyl]trialkylammonium salt, [2-(acryloyloxy)ethyl]trialkylammonium salt, [2-(acryloyloxy)ethyl]trimethylammonium chloride (AETMAC), N,N-dimethylaminoalkyl methacrylamide, N,N-dimethylaminoalkyl acrylamide, N,N,N-trimethylaminoalkyl methacrylamide salt, N,N,N-trimethylaminoalkyl acrylamide salt, (vinylbenzyl)trialklyammonium salt, (vinylbenzyl)trimethylammonium chloride (VBTMAC), acrylic acid (AA), methacrylic acid (MAA), itoconic acid (IC), crotonic acid, maleic anhydride, maleic acid, fumaric acid, mono-2-(methacryloyl)ethyl succinate (MAES), 2-acrylamido-2-methyl-1-propanesulfonic acid (AMPS), vinylphosphonic acid (VPA), and mixtures thereof.

70. The method of claim 61, where the reaction to form the polymer includes reacting about 5 mole percent or less of methacrylic acid (MAA), with the remaining mole percent of the polymer being 2-ethyl hexyl methacrylate (EHMA) and methyl methacrylate (MMA) in a ratio of about 75/25 to about 50/50 mole percent EHMA/MMA to form the polymer.

71. The method of claim 70, where the reaction to form the polymer includes reacting about 2 mole percent or less of methacrylic acid (MAA), with the remaining mole percent of the polymer being 2-ethyl hexyl methacrylate (EHMA) and methyl methacrylate (MMA) in a ratio of about 55/45 to about 50/50 mole percent EHMA/MMA to form the polymer.

72. The method of claim 71, where the solvent comprises about 90 to about 100 percent by volume of octane and about 0 to about 10 percent by volume of isopropanol.

73. The method of claim 61, where the polymer is formed from a reaction product of about 0 to about 10 mole percent of the charged component, the remaining mole percent of a hydrophilic component and the hydrophobic component in a ratio of about 60/40 percent to about 80/20 percent.

74. The method of claim 73, where the hydrophilic component is a material comprising a polymerizable monomer, oligomer, or polymer.

75. The method of claim 74, where the material for the hydrophilic component is selected from the group consisting of N-vinylpyrrolidone (VPL), 2-hydroxyethyl acrylate (HEA), 2-hydroxylethyl methacrylate (HEMA), N,N-dimethyl acrylamide (DMA), N-[tris(hydroxymethyl)methyl]acrylamide (THMMAM), polyethyleneglycol acrylate (PEGA), polyethyleneglycol methacrylate (PEGMA), and mixtures thereof.

76. The method of claim 75, where the reaction to for the polymer includes reacting about 2.0 to about 7.5 mole percent of an acrylic acid (AA) for the charged component, with the remaining mole percent of the polymer being methyl acrylate (MA) for the hydrophobic component, and 2-hydroxyethyl methacrylate (HEMA) for the hydrophilic component in a ratio of about 40/60 percent MA/HEMA to about 20/80 percent MA/HEMA to form the polymer.

77. The method of claim 76, where the solvent comprises about 60 to 85 percent by volume of water and about 15 to 40 percent by volume of ethanol.

78. The method of claim 76, where the solvent further comprises ammonium hydroxide, 2-aminomethylpropanol (2-AMP), and mixtures thereof.

79. The method of claim 61, including using the film forming composition to form a liquid bandage.

80. The method of claim 61, where the solvent has a boiling point less than 75° C. at 1 atm.

81. The method of claim 80, where the solvent comprises about 90 to about 100 weight percent of one or more alkanes and about 0 to about 10 weight percent of one or more non-aqueous polar solvent.

82. The method of claim 81, where the solvent comprises about 90 to about 100 weight percent of one or more alkanes selected from the group consisting of pentane, hexane, isooctane or mixtures thereof, and about 0 to about 10 weight percent of one or more non-aqueous polar solvent selected from the group consisting of an alcohol, ethyl acetate, acetone or mixtures thereof.
